(19) United States
(12) Patent Application Publication
(10) Pub. No.: US 2012/0184044 A1
(43) Pub. Date: Jul. 19, 2012

(54) MOLECULARLY-IMPRINTED POLYMERIC MATERIALS FOR VISUAL DETECTION OF EXPLOSIVES

(75) Inventors: Aristotle G. KALIVRETENOS, Columbia, MD (US); Kelly A. VAN HOUTEN, West Friendship, MD (US); Jonathan P. GLUCKMAN, Arnold, MD (US); Frank M. HARDY, Vienna, VA (US); Igor P. DOROVSKOY, Columbia, MD (US); Robert TROWER, Perryville, AR (US)

(73) Assignee: RAPTOR DETECTION, INC., Columbia, MD (US)

(21) Appl. No.: 12/649,858

(22) Filed: Dec. 30, 2009

Related U.S. Application Data
(60) Provisional application No. 61/141,748, filed on Dec. 31, 2008.

(51) Int. Cl. G01N 33/22; C08F 222/40; C08K 5/05; C08G 63/91

(52) U.S. Cl. 436/106; 525/418; 526/262; 524/379

(57) ABSTRACT

A molecularly-imprinted polymeric material that selectively binds with an explosive compound. The molecularly-imprinted polymeric material comprises a cross-linked, water-soluble polymer having basic functional groups and a binding site capable of selectively binding an explosive compound. The basic functional groups have a pKa that is sufficiently high to react with the explosive compound to result in a visually detectable color change. For example, the basic functional groups may have a pKa in the range of 6.0-9.0. The molecularly-imprinted polymeric material may be used in a variety of applications, such as a projectile for detecting explosives. Also described is a method for making a molecularly-imprinted polymeric material.

### TECHNICAL FIELD

The present invention relates to molecularly-imprinted polymers.

### BACKGROUND

A variety of techniques have been attempted or proposed for using molecularly-imprinted polymers (MIPs) in the detection of explosives. However, many of these known techniques have certain disadvantages. Thus, there is a continuing desire for improved techniques for using MIPs in the detection of explosives.

### SUMMARY

In one aspect, the present invention provides a molecularly-imprinted polymeric material comprising: (a) a cross-linked, water-soluble polymer having basic functional groups; and (b) a binding site capable of selectively binding a high-explosive nitroaromatic compound; wherein the basic functional groups have a pKa that is sufficiently high to react with the explosive compound to produce a visually detectable color change.

In another aspect, the present invention provides a method for detecting a high-explosive nitroaromatic compound, comprising: providing a molecularly-imprinted polymeric material; and contacting the polymeric material with a sample potentially containing a high-explosive nitroaromatic compound.

In another aspect, the present invention provides a method for making a molecularly-imprinted polymeric material, comprising: (a) providing a solution mixture comprising: a template compound; a water-soluble monomer; a basic monomer having a basic functional group with a pKa that is sufficiently high to react with a high-explosive nitroaromatic compound to produce a visually detectable color change; a cross-linking monomer; (b) polymerizing the monomers to form cross-linked, water-soluble polymers that are non-covalently linked to the template compound; and (c) removing the template compound from the water-soluble polymers.

### BRIEF DESCRIPTION OF THE DRAWINGS

FIGS. 1A-1D show a possible reaction scheme by which molecularly-imprinted polymeric materials of the present invention may be made.

FIGS. 2A-2C show a possible reaction scheme by which TNT may undergo a color reaction change. FIG. 2A shows the TNT bound to the polymer. FIG. 2B shows the TNT being converted to an anion by deprotonation of the methyl group. FIG. 2C shows the TNT forming a Meisenheimer complex with the polymer.

FIGS. 3A and 3B shows a projectile loaded with a molecularly-imprinted polymeric material. FIG. 3A shows the projectile traveling towards a target board. FIG. 3B shows the dispersion of the polymeric material onto the target board upon projectile impact.

FIGS. 4A and 4B show cotton T-shirts after test firing of projectiles loaded with a molecularly-imprinted polymeric material of the present invention.

### DETAILED DESCRIPTION

The present invention provides molecularly-imprinted polymeric materials that are designed to selectively bind with hazardous materials, such as high-explosive compounds, thereby detecting the presence of the hazardous material. Upon the binding of the hazardous material, the polymeric material and the hazardous material react with each other to produce a color change that can be directly observed by visualization. As such, the color change may be detected without the need for special equipment (e.g., a spectrometer) or the aid of an intervening processing step (e.g., conversion of color change into an electronic signal that is processed by an interpreting device).

As used herein, the term “molecularly-imprinted polymeric material” refers to synthetic, polymeric mold-like structures that have pre-organized interactive moieties that complement the binding sites on a target hazardous material. The interactive moieties have functional characteristics and a geometric organization which allow the polymeric material to selectively bind the target hazardous material. In addition to explosives, other examples of hazardous materials as targets in the present invention include poisonous gases and lethal biologic agents.

In one aspect, the present invention provides a method of forming such polymeric materials by template-directed synthesis. The method involves polymerizing one or more water-soluble monomers and one or more basic monomers in the presence of one or more template compounds. Via their functional groups, the monomers interact with the template compounds in solution to form a template-monomer complex. The monomers are polymerized with one or more cross-linking monomers to result in cross-linked, water-soluble polymers that are complexed with the template compound. The template compounds are then extracted from the cross-linked polymers to result in a molecularly-imprinted polymeric material that can be used for selectively binding a target hazardous material of interest.

In the case where the target material is an explosive compound, the template compound may be the explosive compound of interest itself, or it may be a non-explosive structural analog of the explosive compound. Examples of high-explosive compounds that may be targeted for detection include nitroaromatic explosive compounds such as trinitrotoluene (TNT), trinitrobenzene (TNB), or tetryl (2,4,6-trinitrophenyl-N-methylnitramine). Other examples of high-explosive compounds include nitrate explosives, such as urea nitrate or guanidine nitrate.

The term “structural analog,” as used herein, refers to a compound that shares molecular structural characteristics with an explosive compound of interest such that a molecularly-imprinted polymeric material that is imprinted with the structural analog will selectively bind with the explosive compound of interest. Non-explosive structural analogs of TNT have an acidic group (e.g., carboxylic acid) such that it can form a salt with the basic monomer(s). In this way, the monomers are held in place during polymerization. For example, non-explosive structural analogs of TNT include TMBA (2,4,6-trimethylbenzoic acid) and TCBA (2,4,6-trichlorobenzoic acid). Other structural analogs of TNT include benzoic acid derivatives having 1 to 3 substituents at the 2, 4, and/or 6 positions of the phenyl ring. These substituents should be similar in size to a nitro group and may be small aliphatic, halogen, or other electron withdrawing groups (e.g., methyl, ethyl, trifluoromethyl, etc.).

Non-explosive structural analogs of TNT also include nitroaromatics that contain only one or two nitro groups, such that it may avoid forming an irreversible Meisenheimer complex with the monomers. Examples of such structural analogs include nitrobenzene, ethylnitrobenzene, ethyldinitrobenzene, dinitrobenzene, nitrotoluene, dinitrotoluene, nitroxylene, dinitroxylene, 4-nitrophenol, and 2,4-dinitrophenol.

The water-soluble monomer may be any polymerizable monomer having a single polymerizable group (e.g., a vinyl group) and a solubility of at least 10 mg/ml in water. The water-soluble monomers may have functional groups capable of interacting with the template compound. Examples of such functional groups on the water-soluble monomers include amines, hydroxyls, carboxyls, sulfhydryls, metal chelates, or combinations thereof.

The water-soluble monomer used in the present invention may be any suitable water-soluble monomer known to be useful for making molecularly-imprinted polymers. Examples of suitable water-soluble monomers include acrylates, such as methylmethacrylate and other alkyl methacrylates. Other examples of water-soluble monomers suitable for use in the present invention are described in U.S. Pat. No. 6,872,786 (Murray et al.), which is incorporated by reference herein.

The basic monomer may be any suitable polymerizable monomer having a single polymerizable group (e.g., a vinyl group) and having one or more basic functional groups. The basic functional groups may be amides or N-heterocycles, such as imidazoles, pyridines, quinolines, etc. As such, examples of basic monomers include vinylpyridines, vinylamides, and vinylimidazoles (e.g., N-vinylimidazole). Other examples of suitable basic monomers include N,N-dialkylaminoalkyl(meth)acrylates (e.g., dimethylaminoethyl acrylate).

In certain embodiments, the basic functional groups have a pKa in the range of 6.0-9.0. This feature may be useful in providing basic functional groups that are basic enough to deprotonate an explosive compound (e.g., nitroaromatics such as TNT or TNB) or form a Meisenheimer complex with the explosive compound, yet not so strongly basic that the resulting polymeric material would interact non-selectively with potential interferents.

The water-soluble and/or basic monomers may interact with the template compound through any of various types of non-covalent bonding mechanisms, including ionic, hydrophilic/hydrophobic, steric, electrostatic, hydrogen bonding, van der Waals forces, metal coordination, or combinations thereof. In some cases, the template compound forms a salt with the basic monomers.

The cross-linking monomers contain two or more polymerizable groups (e.g., multiple vinyl groups) which can take part in a polymerization process. The cross-linking monomers allow the formation of inter-connections within different polymer chains and/or intra-connections within a polymer chain to form a cross-linked polymer.

The reaction may use any suitable cross-linking monomer that is known to be useful for making molecularly-imprinted polymers. Examples of suitable cross-linking monomers include ethyleneglycol dimethacrylate (EDMA), polyethyleneglycol dimethacrylate (PEGDMA), trimethyloylpropane trimethacrylate (TRIM), and divinylbenzene (DVB).

The relative amounts of each of the above-described monomers will vary depending upon the desired chemical or physical properties of the polymeric material. Increasing the relative amount of the basic monomers may yield polymeric materials with more rapid colorimetric reaction kinetics. However, providing too much of the basic monomers can result in loss of selectivity. As such, in some cases, the basic monomers may be provided in an amount in the range of 0.5-15 wt % (relative to the other reactants in the mixture), and in some cases, in the range of 1-10 wt %.

The amount of water-soluble monomer in the mixture is sufficient to form water-soluble polymers. Balanced against the need for basic monomers that provide the colorimetric reaction, in some cases, a suitable ratio (by weight) of the water-soluble monomer to basic monomer may be in the range of 5:1 to 50:1; and in some cases, in the range of 10:1 to 30:1.

The cross-linking monomers are provided in an amount sufficient to cross-link the polymers to provide structural support and stability to the polymer. However, providing too much cross-linking monomers may result in polymers that are insoluble. As such, in some cases, the amount of cross-linking monomers in the reaction mixture is limited to 25 wt % (relative to the other reactants in the mixture) or less; and in some cases, 15 wt % or less; and in some cases, 10 wt % or less.

The polymerization reaction may be carried out in any conventional fashion (e.g., free radical polymerization initiated by UV irradiation or a free radical initiator such as azobisisobutyronitrile (AIBN)). In some cases, the polymerization may be a controlled free radical polymerization process to control the morphology, topology, and/or molecular weight distribution of the polymers (e.g., to a more narrow distribution). For example, the polymerization process may be carried out as a reversible addition fragmentation chain transfer (RAFT) process using a chain transfer agent (i.e., a RAFT reagent). Any of the various types of RAFT reagents known in the art, including dithioester agents, may be used in the RAFT process.

After the polymerization, the polymers may be further processed for purification, separation, isolation, and/or template removal. This processing may be performed in one or more steps, including filtration, centrifugation, washing, chromatographic separation, electrophoresis, and/or dialysis. Template removal may also be facilitated by a change in the pH or ionic strength of the solution.

In some embodiments, purification of the polymers includes subjecting it to a series of precipitations followed by isolation (e.g., by filtration or centrifugation). In some embodiments, purification of the polymers includes dialysis of the polymers. Dialysis may be useful in circumstances where the polymers are highly soluble in water and the template compound cannot be removed by conventional washing techniques (e.g., in the case of TNT). Where dialysis is used, the dialysis membrane selected for use is impermeable to the polymers (e.g., using a dialysis membrane with a molecular weight cut-off that is well below the molecular weight of the polymers). Dialysis can be used to remove all the low molecular weight materials, such as unreacted monomers and the template compound.

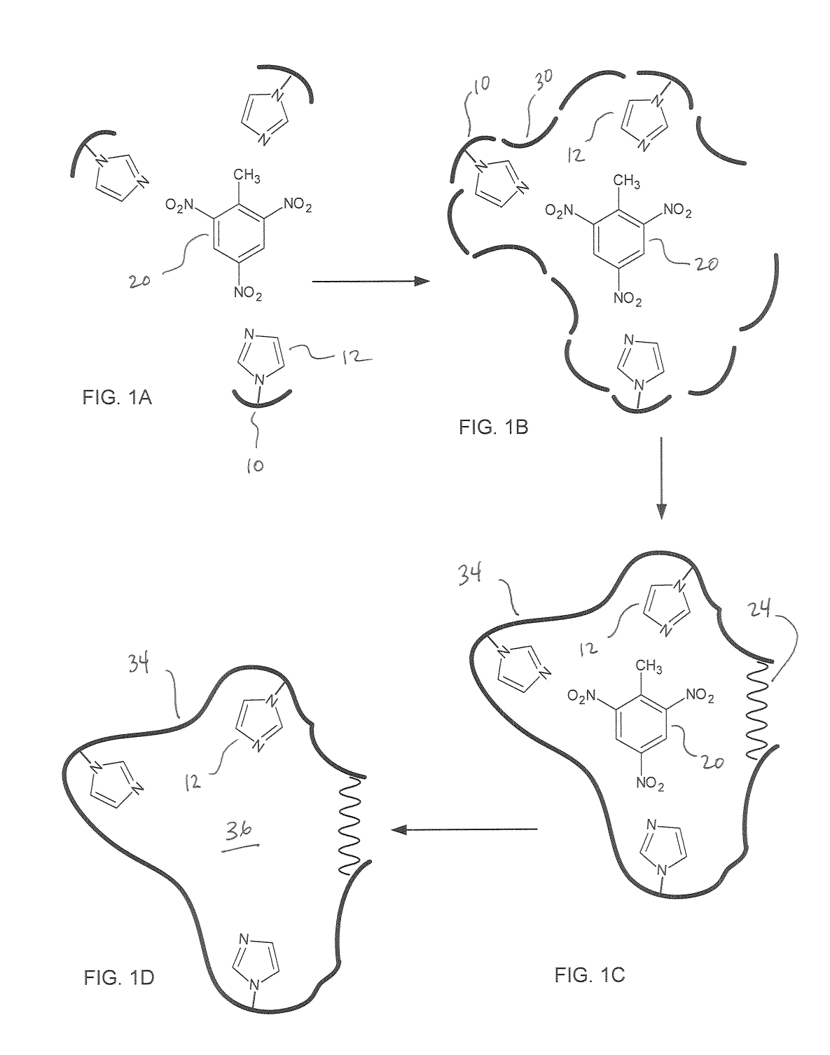

Without intending to be bound by theory, FIGS. 1A-1D show one possible reaction scheme by molecularly-imprinted polymeric material may be formed by the present invention. Referring to FIG. 1A, the basic monomers 10 having imidazole functional groups 12 engage with a TNT molecule 20 in solution. Referring to FIG. 1B, along with water-soluble monomers 30, the basic monomers 10 form a complex with the TNT molecule 20. The monomers then undergo co-polymerization in the presence of a cross-linker. Referring to FIG. 1C, the polymerization results in a polymeric material comprising a polymer 34 having cross-links 24. Furthermore, the polymeric material has a binding site 36 lined with imidazole functional groups 12 that interact with the TNT molecule 20. Subsequently, as shown in FIG. 1D, the TNT molecule 20 is extracted from the polymeric material, leaving behind a binding site 36 having a size, shape, and functional group arrangement for re-binding a TNT molecule.

In another aspect, the present invention provides molecularly-imprinted polymeric materials that selectively bind with a hazardous material, such as a high-explosive compound. The polymeric materials may be made using any suitable synthesis method, including the methods described herein. The polymeric material has binding sites that selectively bind the hazardous material.

The polymeric material comprises cross-linked, water-soluble polymers having one or more basic functional groups that line the binding sites. As used herein, the term “water-soluble polymer” means a polymer having a solubility of at least 10 mg/ml in water. The cross-linking may be intra-connections within a polymer chain or inter-connections between different polymer chains.

The basic functional groups on the polymers interact with the hazardous material through any of various types of non-covalent bonding mechanisms, including ionic, hydrophilic/hydrophobic, steric, electrostatic, hydrogen bonding, van der Waals forces, or combinations thereof. Further, the basic functional groups have a pKa that is sufficiently high to react with the hazardous material to produce a color change reaction. In some cases, the basic functional groups have a pKa in the range of 6.0-9.0. This feature may be useful in providing basic functional groups that are basic enough to deprotonate an explosive compound (e.g., nitroaromatics such as TNT or TNB) or form a Meisenheimer complex with the explosive compound, yet not so strongly basic that the polymeric material would interact non-selectively with potential interferents.

These color change reactions are facilitated when there is a substantial difference between the pKa of the basic functional group and the pKa of the explosive compound. As such, in some embodiments, for a given explosive compound of interest, the basic functional groups are selected such that the pKa difference between the basic functional groups and the explosive compound is at least 3.0; and in some cases, at least 4.0

The polymeric materials may have a variety of chemical or physical properties (e.g., morphology, porosity, solubility, hydrophilicity, stability, etc.), depending on its composition and how its made. For example, such properties can be controlled by the amount of cross-linking, the type of cross-linker used, the strength and amount of the basic functional groups, and whether or not the polymerization process used a RAFT agent.

The morphology of the polymeric materials will also vary, depending upon the particular application. In some cases, the polymeric material may be a bulk, water-soluble polymer matrix formed from a network of cross-linked polymers, with the bindings sites located on or within the polymer matrix.

In some cases, the polymeric material may be individual water-soluble macromolecules with cross-linked binding sites. Such individual macromolecules may have a size in the range of 3,500-200,000 daltons. For example, in some cases, the polymeric material may be polymers having a star-core configuration, in which a number of water-soluble chains extend from a core.

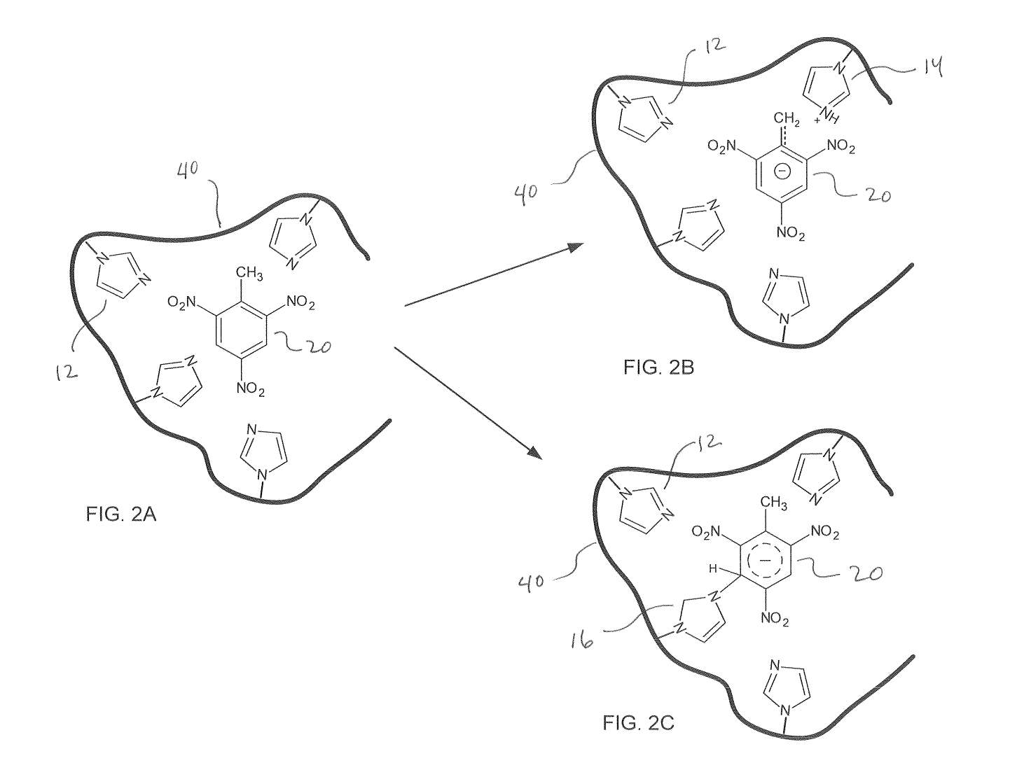

Referring to the embodiment shown in FIGS. 2A-2C, a molecularly-imprinted polymeric material comprises a polymer 40 that forms a binding site lined with imidazole functional groups 12. As shown in FIG. 2A, the imidazole functional groups are positioned at locations where they can align with nitro functional groups on the TNT molecule 20. The imidazole functional groups on polymer 40 may interact with the nitro groups on the TNT molecule 20 via hydrogen bonding or electrostatic attraction.

Without intending to be bound by theory, FIGS. 2B and 2C show two possible reaction mechanisms by which the TNT undergoes a color change. In FIG. 2B, subsequent to bonding of the TNT molecule 20, an imidazole functional group 14 on polymer 40 deprotonates the methyl group on the TNT molecule 20. This deprotonated TNT 20 changes to a red or orange color.

In FIG. 2C, subsequent to binding of TNT molecule 20, the nitrogen on an imidazole functional group 16 makes a nucleophilic attack on the aromatic ring of the TNT molecule 20. As such, the imidazole forms an adduct with the TNT molecule 20, resulting in a Meisenheimer complex that is red or orange colored.

The molecularly-imprinted polymeric materials of the present invention may be provided in a variety of formulations, depending upon the particular application. In some cases, a fluid or gel formulation of the molecularly-imprinted polymeric materials may be provided. For example, in certain embodiments, the molecularly-imprinted polymeric materials may be provided in a solution of a non-organic solvent (e.g., aqueous, aqueous-alcohol, or high purity alcohol, such as a neat alcohol solution). This feature may be useful for applications that are not compatible with organic solvents. For example, the molecularly-imprinted polymeric material may be provided in a container made of a plastic material which would dissolve in an organic solvent.

In some cases, the solvent may be an alcohol, such as methanol or ethanol. This feature may be useful because many explosive compounds (e.g., TNT or TNB) are more readily soluble in alcohol solvents than in water. As such, providing an alcohol formulation may be useful in allowing faster reaction kinetics with the explosive compound.

The amount of polymeric material in the formulation will vary depending upon the particular application. Greater detection resolution may be possible with increasing amounts of polymeric material in the formulation. Greater detection resolution may be beneficial where visual observation is performed from a distance (e.g., when projectiles are used to deliver the polymeric materials). However, large amounts of polymeric material in the formulation may lead to insolubility or increased viscosity.

As such, for molecularly-imprinted polymeric materials made using a non-RAFT process, in some cases, the molecularly-imprinted polymeric materials constitute between 10-50% (weight/weight) of a liquid or gel formulation. For molecularly-imprinted polymeric materials made using a RAFT process, in some cases, the molecularly imprinted polymeric materials constitute between 0.5-3.0% (weight/volume, which refers to the amount of solute in grams as a percentage of the volume of the solution in milliliters). This feature may be useful where the polymeric materials are used in projectiles, which break upon impact, causing dispersal of the molecularly-imprinted polymeric materials. Such formulations may allow visual detection from a distance (e.g., greater than 30 meters), while having sufficiently low viscosity to allow for effective dispersal upon projectile impact.

The molecularly-imprinted polymeric materials of the present invention may be used in a variety of applications for the detection of hazardous materials, such as explosives. For example, the polymeric materials may be applied on a fabric (such as a wipe), loaded into a projectile, contained in a grenade, or contained in a spray apparatus (such as a hand-held spray bottle).

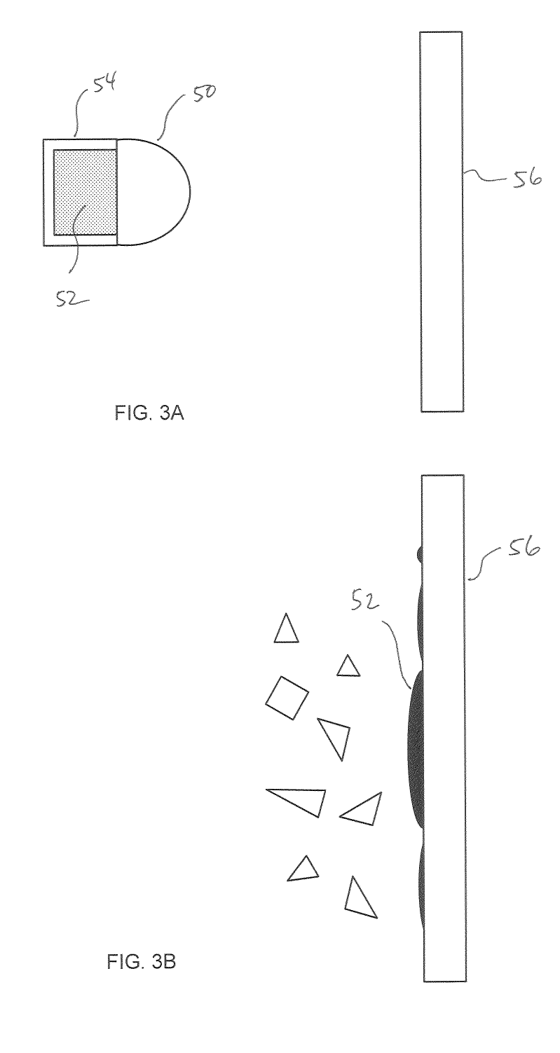

Referring to the embodiment shown in FIGS. 3A and 3B, a projectile 50 has a compartment 54 containing a molecularly-imprinted polymeric material 52 of the present invention. To facilitate shattering upon impact, the shell of projectile 50 is made of polystyrene, which is not compatible with organic solvents. Thus, the molecularly-imprinted polymer material 52 is formulated as an aqueous solution. Projectile 50 is shot at a target board 56 having a sample of TNT smeared on it. As shown in FIG. 3B, upon impact against the target board 56, projectile 50 shatters and splatters the aqueous solution of molecularly-imprinted polymeric material 52 onto target board 56, producing a color change upon detection of the TNT.

### EXAMPLES

Specific representative embodiments of the invention will now be described, including how such embodiments may be made. It is understood that the specific methods, materials, conditions, process parameters, apparatus, and the like, do not necessarily limit the scope of the invention.

Various different approaches were considered for making the molecularly-imprinted polymeric materials of the present invention:

### Approach A: Multi-Step RAFT Process Using Covalently-Linked TNT

In the multi-step approach, functionalized polymers were synthesized by reacting a RAFT reagent (trimethylolpropane tris[3-dithiobenzoyloxypropionate]), an amine-containing monomer, and a methacrylate co-monomer. The polymerization reactions were carried out in a conventional fashion using AIBN as a catalyst. The polymers were purified by dialysis (with MW cutoff of 3,500) to yield polymers as the hydrochloride salt or the free amine-containing polymer.

For polymer imprinting, TNT was covalently linked to the polymer and the polymer was then cross-linked using succinic acid and adipic acid. After additional dialysis, the polymers were then capped by converting unreacted amine groups to amides, thereby reducing non-selective binding. However, the TNT template could not be successfully removed using a series of basic hydrolysis steps.

### Approach B: Single-Step RAFT Process Using Non-Covalent Explosive Template

Functionalized polymers were made by reacting 20 mg of the RAFT reagent; 25 mg (5%) of N-vinylimidazole as a preformed salt with TNT, TNT-acid, or TNB; ˜400 mg of TMAMA (trimethylammonium ethylmethacrylate chloride, a water-soluble monomer); and either 25 mg (5%) or 50 mg (10%) of PEGDMA (polyethyleneglycol dimethacrylate, MW 550) as a cross-linker. The polymerization reactions were carried in sealed tubes with 2 mL dimethylsulfoxide (DMSO) as solvent and AIBN (1%) as catalyst. The mixture was heated at 70-80° C. for 4 hours to promote polymerization.

For isolation and purification of the polymers, the reaction mixtures were dialyzed (MW cutoff of 3,500) against water. For template removal, the polymer solutions were further dialyzed against a sodium bicarbonate buffer and aqueous ammonium solutions. There was limited success in removing the TNT-acid from the polymer, but TNT and TNB appeared to be irreversibly linked to the polymer.

### Approach C: Single-Step RAFT Process Using Non-Covalently Linked Analog.

In another approach, in response to concerns about the hazards of using explosive compounds as template molecules, non-explosive structural analogs of TNT were selected for use as the template. Two different non-explosive templates that form a salt with N-vinylimidazole were used: TMBA (2,4,6-trimethylbenzoic acid) and TCBA (2,4,6-trichlorobenzoic acid).

The functionalized polymers were made by reacting 60 mg of the RAFT reagent, 45 mg (3%) of N-vinylimidazole, ˜1200 mg of TMAMA, 45 mg (3%) of PEGDMA as a cross-linker, and the template compound (either 150 mg of TMBA or 180 mg of TCBA). The polymerization reactions were carried out in sealed tubes with 3 mL dimethylsulfoxide (DMSO) as solvent and AIBN (1%) as catalyst. The reaction mixture was heated at 70-80° C. for 4 hours to promote polymerization.

For isolation and purification of the polymers, along with template removal, the reaction mixtures were dialyzed (MW cutoff of 3,500) against 0.5 M aqueous sodium bicarbonate, followed by water to yield the polymers as pink solids after removal of water under vacuum. The yield for the TMBA-templated polymer was ˜900 mg (67%) and the yield for the TCBA-templated polymer was ˜800 mg (59%).

Various paint formulations were made using the polymers. For the TMBA-templated polymers, 2.5% (w/v) of the polymer was dissolved in a 95:5 propylene glycol/water solution. This 2.5% w/v solution was quite viscous. Also prepared were 1.25% and 0.625% (w/v) formulations in a 90:10 propylene glycol/water solution. Likewise, for the TCBA-templated polymers, 2.5%, 1.25%, and 0.625% (w/v) of the polymer was dissolved in a 90:10 propylene glycol/water solution. Against, the 2.5% w/v solution was quite viscous. Weight/volume (w/v) refers to the amount of solute in grams as a percentage of the volume of the solution in milliliters.

The paint formulations were then tested for sensitivity and selectivity in target detection. The following materials were applied to a paper surface: solid TNT, solid TNB, an aliquot of tetryl in acetonitrile that was allowed to dry, and a number of potential interferents in their purchased state. As used herein, the term “interferents” refers to materials present in a sample that are not the explosive compound(s) that is targeted for detection and that, preferably, would be differentiated from the explosive compound(s) of interest. A small aliquot of test paint was applied to each material and physically mixed with a spatula for about 5 seconds, followed by observation as recorded in Table 1 below.

TABLE 1
Results of target detection tests.

| | 2.5% TMBA-templated | 2.5% TCBA-templated |
|---|---|---|
| Test Sample | polymer formulation | polymer formulation |
| Control (no explosive added) | No color change. | No color change. |

Explosives

| | | |
|---|---|---|
| TNT | Red brown initial color. Fully | Red brown initial color. Fully |
| | develops over ~ 30 mins. | develops over ~ 30 mins. |
| TNB | Orange initial color. Fully | Orange initial color. Fully |
| | develops over ~ 30 mins. | develops over ~ 30 mins. |
| Tetryl | Yellow-orange initial color. | Yellow-orange initial color. |
| | Fully develops over ~ 30 | Fully develops over ~ 30 |
| | mins., then fades slowly over | mins., then fades slowly over |
| | time. | time. |

Interferents

| | | |
|---|---|---|
| ALL ® Small & Mighty | No change. | No change. |
| Free/Clear laundry detergent | | |
| BAND-AID ® anti-itch gel | No change. | No change. |
| BARBASOL ® shaving cream | No change. | No change. |
| CLEARSIL ® acne treatment | Faint yellow. Color develops | Faint yellow. Color develops |
| cream | fast. | fast. |
| COLGATE ® regular | No change. | No change. |
| toothpaste | | |
| COPPERTONE ® sunscreen | Faint yellow. Color develops | Faint yellow. Color develops |
| lotion, SPF 50 | slowly over minutes. | slowly over minutes. |
| COPPERTONE ® sunscreen | Faint yellow. Color develops | Faint yellow. Color develops |
| lotion, SPF 30 | slowly over minutes. | slowly over minutes. |
| BLISTEX ® medicated lip | Very faint yellow. Color | Very faint yellow. Color |
| balm, SPF 15 | develops slowly over minutes. | develops slowly over minutes. |
| IVORY ® bar soap | No change. | No change. |
| OFF! ® insect repellant | No change. | No change. |
| PERT PLUS ® medium | No change. | No change. |
| shampoo | | |
| SCOPE ® original mint | No change. | No change. |
| mouthwash | | |
| SPEED STICK ® regular | No change. | No change. |
| deodorant | | |
| Diesel fuel #2 | No change. | No change. |

These results demonstrate that the polymer paint formulations are capable of target compound detection with both high sensitivity and high selectivity. In many cases, there was an initial color change that developed into a deeper color over time. Even faster color development may be possible when used in projectiles due to the dynamic forces of impact.

For sensitivity evaluation, the 2.5% TMBA-templated polymer paint formulation was tested for TNB detection at various concentrations. The results, as shown in Table 2 below, demonstrate that the polymer formulations are sufficiently sensitive for detecting explosive compounds are relatively low concentrations.

TABLE 2
Sensitivity of TMBA-templated polymer
paint formulation.

| TNB concentration | Color |
|---|---|
| 20 μg/cm² | Orange initial color. Fully |
| | develops over ~ 2 hrs. |
| 5 μg/cm² | Orange initial color. Fully |
| | develops over ~ 2 hrs. |
| 1 μg/cm² | Orange initial color. Fully |
| | develops over ~ 2 hrs. |

### Approach D: Non-RAFT Process Using Non-Covalently Linked Analog.

Water-soluble non-RAFT polymers were made by using a process analogous to Approach C above, except without the RAFT reagent, and using trimethylammonium ethylacrylate chloride as the water-soluble monomer and polyethyleneglycol diacrylate as the cross-linker The resulting polymers were purified by either filtration or dialysis (MW cutoff of 3,500) against a 0.5 M sodium bicarbonate buffer followed by water. Various different paint formulations were made using the non-RAFT polymers, as shown in Table 3 below.

TABLE 3
Non-RAFT polymer paint formulations.

| % N-vinyl- | Formulation composition | | | | |
|---|---|---|---|---|---|
| imidazole | (in % w/w, relative to weight of solvent) | | | | |
| (used in | | | Propylene | Isopropyl | |
| reaction) | Polymer | Water | glycol | alcohol | Methanol* |
| 3% | 10% | 10% | 80% | | |
| 3% | 10% | 10% | 70% | 10% | |
| 3% | 10% | 10% | 60% | 20% | |
| 3% | 30% | | | | 70% |
| 3% | 50% | | | | 50% |
| 6% | 30% | | | | 70% |
| 6% | 40% | | | | 60% |

*Liguid methanol has a density of 0.79 g/ml at 1 atm, 20° C.

The non-RAFT polymer paint formulations were then tested for sensitivity and selectivity in target detection in a manner analogous to that described above for the RAFT polymers. The results, as shown in Table 4 below, demonstrate that the non-RAFT polymer formulations exhibited a much faster detection with greater sensitivity than the RAFT polymer formulations.

TABLE 4
Non-RAFT polymer detection tests.

| | | |
|---|---|---|
| | 3% N-vinylimidazole, | 6% N-vinylimidazole, |
| Test Sample | 30% w/w formulation | 30% w/w formulation |

Explosives

| | | |
|---|---|---|
| TNT | Instant brown | Instant brown color. |
| | color. Fully develops | Fully develops |
| | over ~ 30 mins. | over ~ 30 mins. |
| TNB | Instant red/orange color. | Instant red/orange color. |
| | Fully develops | Fully develops |
| | over ~ 30 mins. | over ~ 30 mins. |
| Tetryl | Instant yellow-orange | Instant yellow-orange |
| | color. | color. |
| | Fully develops over ~ 30 | Fully develops over ~ 30 |
| | mins., then fades | mins., then fades |
| | slowly over time. | slowly over time. |

Interferents

| | | |
|---|---|---|
| CLEARSIL ® acne | No initial color | No initial color change. |
| treatment cream | change. Color | Color develops |
| | develops over minutes. | over minutes. |
| COPPERTONE ® | Very faint yellow | Very faint yellow |
| sunscreen | color develops | color develops |
| lotion, SPF 50 | over ~ 30 mins. | over ~ 30 mins. |
| COPPERTONE ® | Faint yellow | Faint yellow |
| sunscreen | color develops | color develops |
| lotion, SPF 30 | over ~ 30 mins. | over ~ 30 mins. |
| BLISTEX ® | Faint yellow | Faint yellow |
| medicated | color develops | color develops |
| lip balm, SPF 15 | over ~ 30 mins. | over ~ 30 mins. |

For sensitivity evaluation, the two non-RAFT formulations were tested for TNB detection at various concentrations. The results, as shown in Table 5 below, demonstrate that the non-RAFT polymer formulations are sufficiently sensitive for detecting explosive compounds are relatively low concentrations.

TABLE 5
Non-RAFT polymers sensitivity tests.

| TNB | 3% N-vinylimidazole, | 6% N-vinylimidazole, |
|---|---|---|
| Concentration | 30% w/w formulation | 30% w/w formulation |
| 20 μg/cm² | Instant red/orange color. | Instant red/orange color. |
| | Fully develops over ~ 30 | Fully develops over ~ 30 |
| | mins. | mins. |
| 5 μg/cm² | Instant red/orange color. | Instant red/orange color. |
| | Fully develops over ~ 30 | Fully develops over ~ 30 |
| | mins. | mins. |

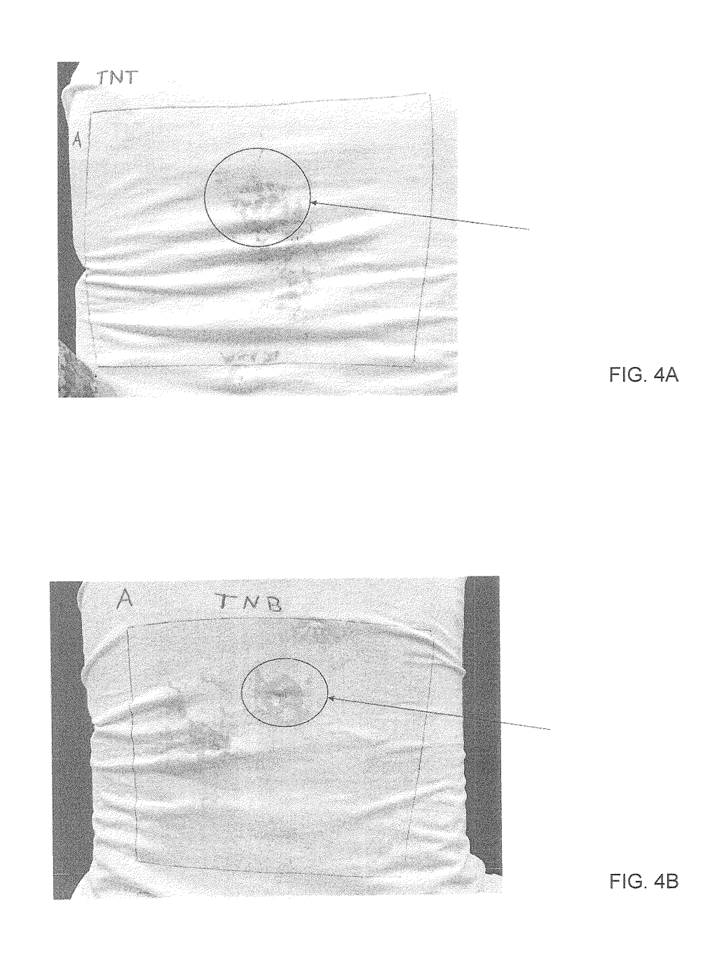

Based on the formulation testing, it is believed that, in the case of non-RAFT polymers, formulations containing 30-50% (w/w) have a level of aqueous solubility, detection resolution, detection kinetics, and viscosity suitable for use in projectile delivery. As such, the 40% non-RAFT polymer/6% imidazole formulation was used in projectile testing. This polymer formulation was loaded into polystyrene projectiles and test fired from a distance of about 25 meters against various targets: cotton, wood panel, metal panel, and cement block. FIGS. 4A and 4B show the results obtained from the test firing on cotton T-shirt targets. The T-shirt shown in FIG. 4A had 5 μg/cm² of TNT applied onto the demarcated area. The T-shirt shown in FIG. 4B had 5 μg/cm² of TNB applied onto the demarcated area. The circles indicate the site of projectile impact.

Alternatively, the non-RAFT polymers may be purified through a series of precipitations. For example, the following series of steps were successfully used for large-scale purification of the polymers. The crude polymer was precipitated with isopropanol (IPA) and isolated by removal of the IPA. The polymer was then further purified by treatment with 0.6 M HCl, followed by precipitation with IPA. The polymer was then further purified by treatment with 0.5 M sodium bicarbonate, followed by precipitation with IPA. The polymer was then further purified by washing with a IPA:water mixture (1:1) and dried to yield a purified polymer product.

The foregoing description and examples have been set forth merely to illustrate the invention and are not intended to be limiting. Each of the disclosed aspects and embodiments of the present invention may be considered individually or in combination with other aspects, embodiments, and variations of the invention. In addition, unless otherwise specified, none of the steps of the methods of the present invention are confined to any particular order of performance. Modifications of the disclosed embodiments incorporating the spirit and substance of the invention may occur to persons skilled in the art and such modifications are within the scope of the present invention.

What is claimed is:

1. A molecularly-imprinted polymeric material comprising:
a cross-linked, water-soluble polymer having basic functional groups; and
a binding site capable of selectively binding a high-explosive nitroaromatic compound;
wherein the basic functional groups have a pKa that is sufficiently high to react with the explosive compound to produce a visually detectable color change.

2. The polymeric material of claim 1, wherein the basic functional groups have a pKa in the range of 6.0-9.0.

3. The polymeric material of claim 1, wherein the basic functional groups are capable of undergoing an acid-base reaction with the explosive compound with deprotonation of the explosive compound.

4. The polymeric material of claim 1, wherein the basic functional groups are capable of forming a Meisenheimer complex with the explosive compound via a nucleophilic substitution reaction.

5. The polymeric material of claim 1, wherein the water-soluble polymer is an individual macromolecule having a molecular weight in the range of 3,500-200,000 daltons.

6. The polymeric material of claim 5, wherein the water-soluble polymer has a star-core configuration.

7. The polymeric material of claim 1, wherein the basic functional groups are imidazole-containing functional groups.

8. A solution containing the molecularly-imprinted polymeric material of claim 1 in a non-organic solvent.

9. The solution of claim 8, wherein the non-organic solvent is an alcohol.

10. The solution of claim 9, wherein the alcohol is methanol or ethanol.

11. The solution of claim 8, wherein the amount of the polymeric material is in the range of 30-50 wt %.

12. An article of manufacture comprising the molecularly-imprinted polymeric material of claim 1.

13. The article of claim 12, wherein the article is a projectile loaded with the molecularly-imprinted polymeric material.

14. A method for detecting a high-explosive nitroaromatic compound, comprising:
using a molecularly-imprinted polymeric material of claim 1; and
contacting the polymeric material with a sample potentially containing a high-explosive nitroaromatic compound.

15. The method of claim 14, wherein the basic functional groups have a pKa in the range of 6.0-9.0.

16. The method of claim 15, wherein the difference in the pKa of the basic functional groups and the pKa of the explosive compound is at least 3.0.

17. The method of claim 14, wherein the polymeric material deprotonates the explosive compound.

18. The method of claim 14, wherein the polymeric material forms a Meisenheimer complex with the explosive compound.

19. A method for making a molecularly-imprinted polymeric material, comprising:
(a) using a solution mixture comprising:
a template compound;
a water-soluble monomer;
a basic monomer having a basic functional group with a pKa that is sufficiently high to react with a high-explosive nitroaromatic compound to produce a visually detectable color change;
a cross-linking monomer;
(b) polymerizing the monomers to form cross-linked, water-soluble polymers that are non-covalently linked to the template compound; and
(c) removing the template compound from the water-soluble polymers.

20. The method of claim 19, wherein the basic functional groups have a pKa in the range of 6.0-9.0.

21. The method of claim 19, wherein the template compound is the high-explosive nitroaromatic compound or a non-explosive structural analog of the high-explosive nitroaromatic compound.

22. The method of claim 19, wherein the mixture further comprises a reversible addition fragmentation chain transfer (RAFT) agent.
